(19) United States
(12) Patent Application Publication
(10) Pub. No.: US 2001/0011128 A1
(43) Pub. Date: Aug. 2, 2001

(54) Hex II tumor-specific promoter and uses thereof

(76) Inventors: Gerald Batist, Montreal (CA); Maha Katabi, Montreal (CA)

Correspondence Address: Nixon Peabody LLP, c/o David S. Resnick, 101 Federal Street, Boston, MA, 02110-1000, US

(21) Appl. No.: 09/739,223

(22) Filed: Dec. 19, 2000

Related U.S. Application Data
(63) Continuation-in-part of application No. 09/276,005, filed on Mar. 25, 1999.
Application No. 09/276,005 is a continuation of application No. PCT/CA97/00691, filed on Sep. 22, 1997.
(60) Provisional application No. 60/026,678, filed on Sep. 25, 1996.

(51) Int. Cl. A61K 48/00; A01N 63/00

(52) U.S. Cl. 536/23.1; 424/93.21; 435/455; 435/320.1

(57) ABSTRACT

The present invention relates to a tumor-specific promoter for use in gene targeted therapy that is selectively activated in cancer cells, which comprises a Hex II promoter. The present invention also relates to a gene construct, which include a Hex II promoter in a vector selected from a basic expression vector, a shuttle plasmid, an adenovirus type 5 recombinant vector and a lipid-based delivery system.

### RELATED APPLICATIONS

This application is a continuation-in-part of U.S. patent application Ser. No. 09/276,005, filed Mar. 25, 1999 which is a continuation of PCT/CA97/00691 filed Sep. 22, 1997, designating the United States and claiming priority right under 35 U.S.C. 119(e) of U.S. Provisional Patent Application Ser. No. 60/026,678 filed Sep. 25, 1996.

### BACKGROUND OF THE INVENTION

(a) Field of the Invention

The invention relates to a novel tumor-specific promoter for use in gene targeted therapy that is differentially regulated in cancer cells, such as to drive a suicide gene in cancer therapy.

(b) Description of Prior Art

A successful gene therapy approach is dependent upon two parameters: 1) efficiency of target cell transduction and 2) specificity of gene delivery. Selective targeting is especially critical in the context of cancer therapy for gene directed enzyme prodrug therapy (GDEPT), where a suicide gene expressed in tumor cells encodes an enzyme that converts an otherwise non-toxic prodrug into its active form.

Several methods have been explored to increase the specificity. They can be broadly divided into two categories: directed delivery of the gene of interest or its directed expression. The ideal candidate for transcriptional targeting would be a tumor specific promoter and/or enhancer and its activation will be strong enough to achieve therapeutic levels of the desired transcript. A wide range of promoters have been explored in this context. They were mostly characterized as tissue specific promoters as opposed to tumor selective. Some examples are: surfactant protein SP-A promoter for non small cell lung carcinoma (NSCLC), immunoglobulin enhancer or O enhancer for B-cell lineage cancers, tyrosinase for melanomas, and MUC-1/Df3 for breast cancer. However, these promoters also direct gene expression in the normal tissue of origin of these neoplasms and other critical organs as well. The erbB2 and a-fetoprotein promoters are activated to a greater extent in certain neoplasms. They have also been used in this strategy and have lead to promising results. Nonetheless, other promoters to further improve and optimize this strategy are needed.

A striking characteristic of rapidly growing tumor cells is their high rate of glucose utilization compared to their normal counterparts. Glucose is mainly channeled through the glycolytic pathway which is not only used for rapid energy production but also for the provision of biosynthetic precursors necessary to sustain a high rate of cellular division. Hexokinase (ATP: D-hexose-6-phosphotransferase) catalyses the first committed step of glycolysis; therefore it was suspected by many to be a potential player in this phenotype. Hexokinases (HK) are comprised of two highly homologous 50 kDa halves and are product inhibited by glucose-6-phosphate to varying degrees. They exist in four molecular forms, HK I to HK IV, with distinct electrophoretic and kinetic properties (Wilson, J. E., (1985) In Regulation of Carbohydrate Metabolism, Vol. I, 45-85, CRC Press, Boca Raton). The profile of these enzymes in tissues at different stages of malignancies shows an increase in HK II in tumor versus normal tissues. In rats, the type I HK is expressed in brain, kidney and heart. The type II HK was found in skeletal muscle and in AH130 hepatoma cells. In normal liver it is type IV HK that is most abundant (Mathupala, S. P., Rempel, A., and Pedersen, P. L. (1995) J. Biol. Chem. 270, 16918-16925).

Mathupala et al. ((1995) J. Biol. Chem. 270, 16918-16925) reported the isolation and sequencing of the rat Hex II promoter from rapidly growing, highly glycolytic hepatoma cell line (AS-30D). Enhanced activity of the rat Hex II promoter in the rat tumor cell line (AS-30D) as compared with transfected rat hepatocytes, in the presence of modulators of interest, i.e. glucose, insulin and glucagon was also reported. Mathupala et al. further reported that differences in the regulation of hexokinase genes involved in glucose catabolism appear between normal versus tumor cells in rat.

Comparison of the rat hexokinase II with a hexokinase from rat Novikoff ascites shows there is a single type II isozyme that is found in both normal and tumor tissues (Adams, V., Kempf, W, Hassam, S., and Briner, J. (1995) Biochem. Mol. Med. 54, 53-58). The inhibition of HK II by glucose-6-phosphate is delayed. Therefore, tumors are able to build up high levels of this product. Its accumulation is a signal for glucose availability for consumption, a stimulus of biosynthetic pathways for growth (Wilson, J. E., (1985) In Regulation of Carbohydrate Metabolism, Vol. I, 45-85, CRC Press, Boca Raton).

Adams et al. also found the level of HK to be increased in human HepG2 cells and in renal cell carcinoma suggesting that a shift in hexokinase isozyme composition in humans may be an ubiquitous phenomenon during malignant transformation.

Despite reported differences in the regulation of rat hexokinase type II promoter in normal rat hepatocytes and rat hepatoma cells and suggestions that hexokinase isozyme composition is an indicator of malignancy, a hexokinase promoter construct having tumor-selective activity has not been previously taught. Further, the selective expression of a rat hexokinase promoter in non-rat cells has not been previously taught nor suggested.

It would be highly desirable to be provided with a novel tumor-specific promoter that is selectively regulated in cancer cells as compared to normal cells. Further, it would be desirable to be provided with a tumor-specific promoter for use in gene targeted therapy to selectively target cancer cells.

### SUMMARY OF THE INVENTION

One aim of the present invention is to provide a novel tumor-specific promoter for use in gene targeted therapy that is differentially regulated in cancer cells, such as to drive a suicide gene in cancer therapy.

In accordance with an embodiment of the present invention there is provided a tumor-specific promoter for use in gene targeted therapy that is differentially regulated in cancer cells, which comprises Hex II reporter gene.

In accordance with the another embodiment of the present invention there is provided a tumor-specific gene construct, which comprises a rat Hex II promoter in a suitable vector, wherein said promoter is selectively activated in tumor cells as compared with normal cells.

In accordance with a further embodiment of the present invention there is provided a vector for use in selective gene expression in a tumor cell, said vector comprising a rat Hex II promoter that is selectively activated in tumor cells as compared with normal cells.

In accordance with yet a further embodiment of the present invention there is provided a method for a tumor-selective expression of a gene in a cell comprising inserting in said cell a gene construct comprising said gene operably linked to a tumor-specific rat Hex II promoter, whereby said rat Hex II promoter is selectively activated in tumor cells as compared with normal cells.

In accordance with another embodiment of the present invention there is provided a tumor-specific Hex II gene construct comprising a rat Hex II promoter operatively linked to a gene and a vector selected from one of a basic expression vector, a shuttle plasmid, an adenovirus type 5 recombinant vector or a lipid-based delivery system.

In accordance with still a further embodiment of the present invention there is provided a kit adapted to provide a tumor-specific gene construct.

In accordance with still a further embodiment of the present invention there is provided a kit adapted to provide a tumor-specific Hex II promoter construct for use in screening tumor-specific gene expression in vitro.

### BRIEF DESCRIPTION OF THE DRAWINGS

FIG. 1 illustrates the Hex II reporter gene construct in pCAT basic expression vector in accordance with an embodiment of the present invention;

FIG. 2 illustrates the Hex II promoter construct including β-galactosidase in the shuttle plasmid pΔ E1sp1B in accordance with another embodiment of the present invention;

FIG. 3 illustrates the Hex II promoter construct including HSV Tk in the shuttle plasmid pΔE1sp1B in accordance with yet another embodiment of the present invention;

FIG. 4 illustrates a graph of the results of MUC-1 versus HexII promoter activation in normal bronchial and mammary epithelial cells;

FIG. 5 illustrates a graph of the results of HexII promoter activation in normal bronchial epithelial cells versus non-small cell lung carcinomas.

FIGS. 6A-6H illustrate the results of histochemical staining on tissue sections of tumor samples injected with recombinant AdHexLacZ and AdΔ E1E3.

FIGS. 7A-7C illustrate tumor growth in DA3 mice treated with Adenovirus constructs in accordance with an embodiment of the present invention;

FIG. 8 illustrates a strategy for generating the HK II promoter reporter gene construct pHexII4557 CAT, and pUC/HexIILacZ and pAdBN/HexLacZ;

FIGS. 9A-9C illustrate the results of promoter activation studies in a panel of normal and tumor cells, expressed as percent acetylation in pHexII4557 CAT of pMUC1-1583 CAT transfectants relative to percent acetylation in pRSV CAT transfectants. Results are the average of three or four independent experiments, with each condition done in duplicate.

FIGS. 10A-10C illustrates regulation of the HK II promoter in normal and transformed cells.

FIGS. 11A-11I illustrate histochemical staining for β-galactosidase expression. 11A-11C: NCI-H661 cells infected with ADΔE1E3, AdHexLacZ or AdCMVLacZ, respectively; 11D-11F: NCI-H460 cells infected with AD ΔE1E3, AdHexLacZ, or AdCMVLacZ, respectively; 11G-11I: NHBECs infected with ADΔE1E3, AdHexLacZ or AdCMVLacZ, respectively;

FIGS. 12A-12C illustrate a dose-response diagram of AdHexTk or AdHexLacZ MOI versus cell killing using two different doses of GCV; Experiments were carried out in quadruplicate for each condition with at least three repeats for every cell line; 12B: illustrates cell-killing curves in several cell lines, showing toxicity over a range of GCV concentrations in AdHexTk-infected cells; and FIG. 12C: illustrates cell-killing curves in several cell lines, showing toxicity over a range of GCV concentrations in AdHexTk-infected cells.

### DETAILED DESCRIPTION OF THE INVENTION

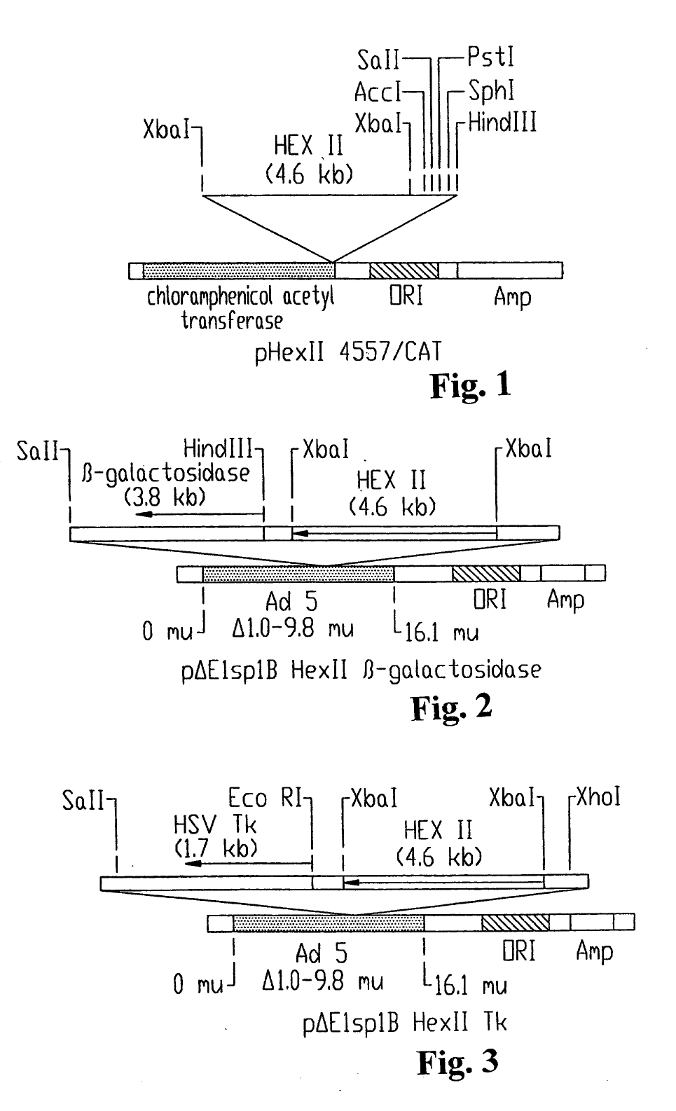

In accordance with the present invention, there is provided a new tumor selective promoter. Examples of constructs comprising the tumor selective promoter of the present invention are illustrated in FIGS. 1 to 3. In particular, a tumor selective rat Hex II promoter is provided. According to an embodiment of the present invention, a rat Hex II promoter is provided that is selectively overexpressed in non-rat tumor cells by transcriptional activation.

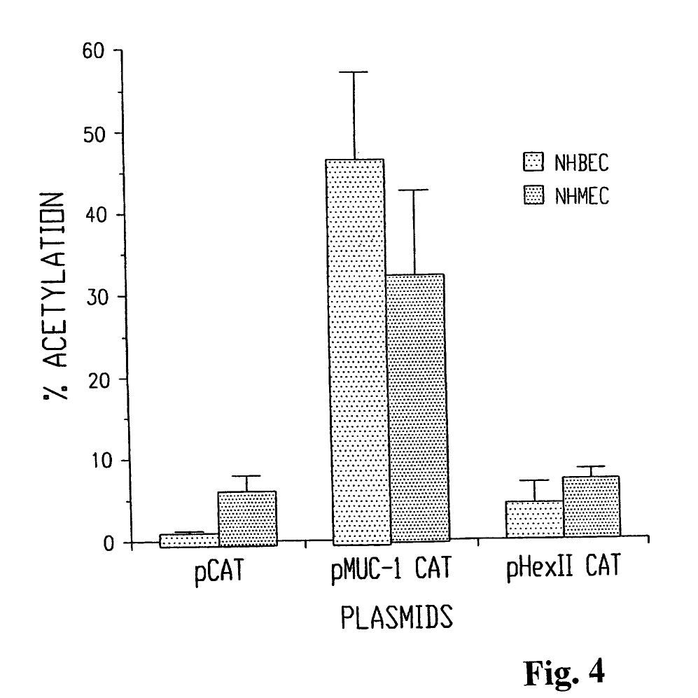

The use of tissue or tumor selective promoters in targeted gene therapy for cancer depends on strong promoters with specific activity. The Muc-1/Df3 promoter has been used in the context of gene directed enzyme prodrug therapy (GDEPT) (Chen et al. (1995) J. Clin. Invest. 96(6), 2775). However we have found that it has limited promoter activity and appears to be expressed in a wide range of normal cells (FIG. 4). An interesting property of cancer cells that could be exploited to target them selectively is their increased rate of glycolysis. Hexokinase type II (Hex II) catalyzes the first committed step of glycolysis and has been linked to this phenotype since it is overexpressed in tumors and is not responsive to the normal physiological inhibitors, e.g. glucagon (Mathupala, S. P., Rempel, A., and Pedersen, P. L. (1995) J. Biol. Chem. 270, 16918-16925).

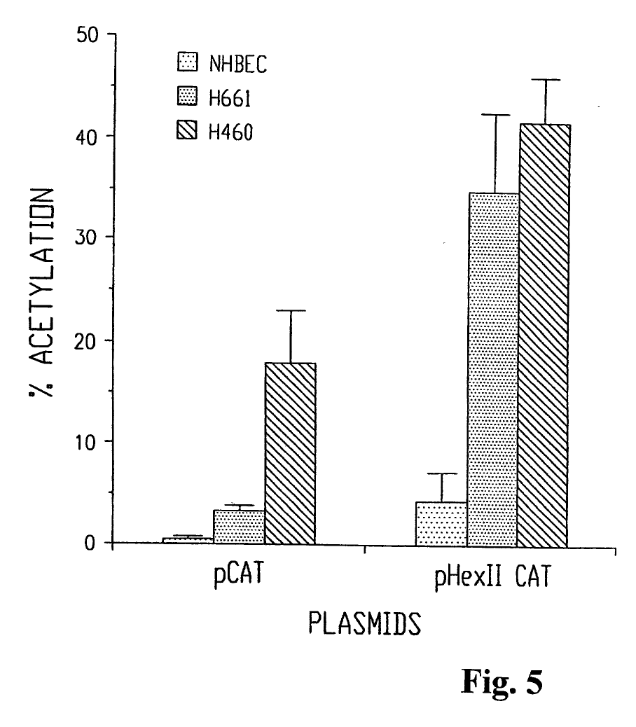

In accordance with the present invention, the tumor HK II promoter was tested in variety of human tumor cell lines and in normal human cells. We studied the Hex II promoter by transfecting cells with the pHex II4557/CAT (FIG. 1) construct and performing a chloramphenicol acetyl transferase (CAT) reporter gene assay. The results of these studies are outlined below, and are provided to exemplify the tumor selective activity of the rat Hex II promoter (FIG. 5). It is fully contemplated that the rat Hex II promoter of the present invention may be delivered to both human and non-human cells using any suitable delivery system known in the art, and is not limited to those examples herein described.

1. Construction of Recombinant Plasmids

pHexII4557-CAT

(8.9 kb) The HexII, 5.15 kb, promoter in the plasmid pUC18 (Mathupala, S. P., Rempel, A., and Pedersen, P. L. (1995) J. Biol. Chem. 270, 16918-16925) was released with an Xbal digest and cloned into the pCAT basic vector (Promega). The size of the promoter was reduced to 4.56 kb with a BamHI digest that released sequences from the non coding region at the 3′ end of the clone.

pΔE1sp1BHex-LacZ

(14.7 kb) the 3.74 kb lacZ gene (HindIII-SalI) from pSV2-β-galactosidase was cloned into the HindIII and SalI polycloning sites of the shuttle vector pΔ E1sp1B. This shuttle plasmid contains Adenovirus 5 (Ad5) sequences from map unit 0 to 1, followed by the polycloning site, followed by Ad5 sequences from mu 9.8 to 15.8, and therefore allows recombination to take place with the adenoviral genome. The Hex II promoter 4557 bp was released from the pHexII 4557/CAT with XbaI followed by an EcoRI digest and cloned into the XbaI site of the pΔE1sp1B. Clone 10 (pΔ E1sp1BHexII) that had the insert in the negative orientation relative to the polycloning site of the pΔ E1sp1B was used for further cloning of the Hex LacZ plasmid. pΔE1sp1BLacZ was digested with XhoI followed with a partial digest with EcoRI. pΔE1sp1BHexII was in turn digested with XhoI and EcoRI, and the purified 4.6 kb fragment was ligated into pΔE1sp1BLacZ.

pΔE1sp1BHex-TK

(12.6 kb) The 1.7 kb HSV-TK gene (EcoRI-SalI) from pMC1TK was cloned into the corresponding sites of pΔE1sp1B. Subsequently, the resulting pΔE1sp1BTK plasmid was cut with EcoRI and XhoI, and the purified 4.6 kb HexII fragment with compatible ends was ligated into it. Plasmid DNA was purified by alkaline lysis followed by cesium chloride density gradient purification.

2. Transfection and Reporter Gene Assays

Transient transfections were performed using lipofectamine according to the manufacturer's recommendations (GIBCO-BRL). Cells were plated the day before transfection to give 60% confluency in 6-well plates. The p1583/+33MUC1.CAT or pHex4557.CAT vectors were transfected along with pSV₂lacZ to determine promoter activity. 1 ug of each plasmid were used for each well. All conditions assayed were done in duplicate. The plasmids pRSV.CAT and promoterless pCAT were used as positive and negative controls, respectively. Cells extracts were prepared 48 hours after transfection and β-galactosidase activity was assayed to compensate for variations in transfection efficiency. CAT activity was determined from 75-100 ug of proteins. The reaction was carried out with 0.1 uCi of ¹⁴C-labeled chloramphenicol in a 100 ul reaction at 37° C. for 4 hrs.

Results

Its activation was very high in tumor as opposed to normal cells. The activation of Hex II in the non-small cell lung carcinomas H661 and H460 was 43% and 64% (respectively) of the activation observed with the Rous Sarcoma virus (RSV) constitutive promoter while it was 3% of RSV in the primary normal human bronchial epithelial cells (NHBEC). Moreover, treatment of the transfectants with glucagon did not inhibit promoter activation in H661 cells. Its activation in the human mammary carcinoma cells MCF-7 was 72% of RSV while it was 23% of RSV in the normal human mammary epithelial cells (NHMEC).

Moreover, the efficacy of this promoter in the context of GDEPT was tested by using the herpes thymidine kinase gene in combination with the prodrug gancyclovir.

In addition, the Hex II promoter of the invention may be operatively linked, in a suitable gene construct, to a suicide gene, antisense oligonucleotide, pro-apoptotic gene or the like and used to selectively express that gene in tumor cells. For example, a Cytochrome P-450™ 2B1 or a HSV TK gene may be used with a Hex II promoter construct of the present invention for selective expression thereof in tumor cells. Accordingly, these genes may be provided together with a corresponding prodrug to effect a toxic response in tumor cells. For example, in the case of HSV TK, the prodrug ganciclovir may be provided, while the prodrugs cyclophosphamide, penicillin, amidase or β-lactamase may be provided together with Cytochrome P-450™ 2B1.

The tumor selective expression of the Hex II promoter of the present invention has utility in both in vivo cancer therapy as well as in vitro laboratory research. As an example, a Hex II promoter construct of the present invention including a suitable gene, such as a suicide gene, operably linked thereto may be used in cancer therapy to target an in vivo tumor and selectively express the suicide gene in tumor cells. Alternatively, for example, a Hex II promoter of the present invention may be employed with a suitable gene operatively linked thereto for screening of tumor cells in vitro.

3. MTT Cell Viability Assays

Cell survival was determined using a colorimetric assay which measures the ability of viable cells to reduce a soluble yellow tetrazolium salt (MTT) to an insoluble purple formazan precipitate. Cells in the logarithmic phase of growth were resuspended at a concentration of 2×10⁵ cells/ml. 2 ml/well were plated in 6-well plates. Plates were incubated for 24 h at 37° C. in 5% CO₂. Subsequently, cells were transfected with the pΔE1sp1B Hex TK plasmid as described above. 6 h after the transfection, cells were treated with the drug gancyclovir at concentrations of 10 or 25 ug/ml. Each condition was done in triplicate. Cells survival was calculated in the treated population as a percentage of controls. Controls are cells transfected with the plasmid alone or treated with the drug alone. MTT assays was performed two days following treatment. The formazan crystals were dissolved in dimethyl sulfide (Fisher) and glycine buffer (0.1 M glycine-0.1 M NaCl, pH 10.5). The formazan product formed by viable cells was quantitated by measuring the absorbance at a wavelength of 570 nm.

Results

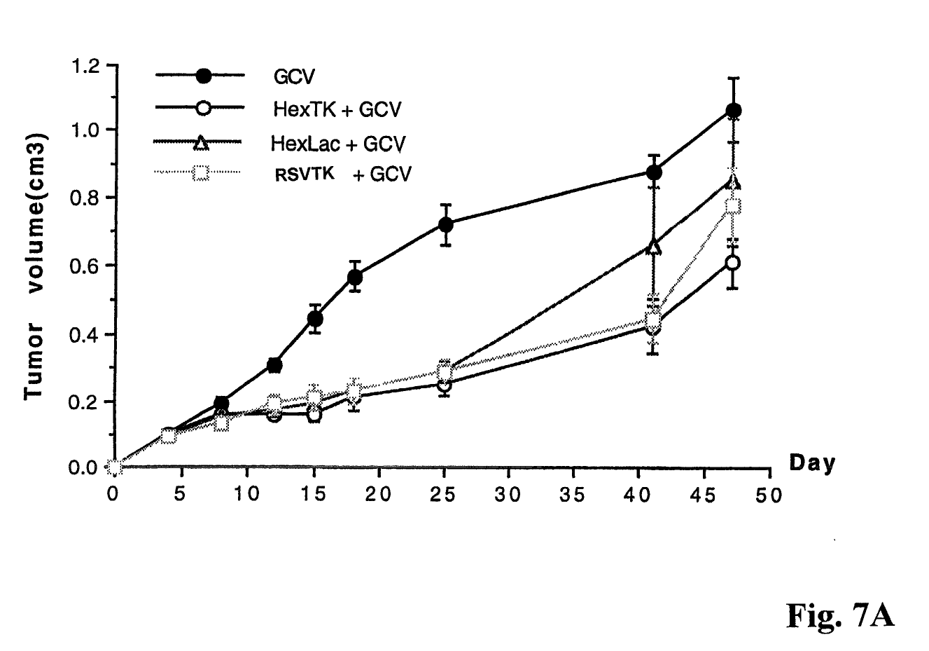

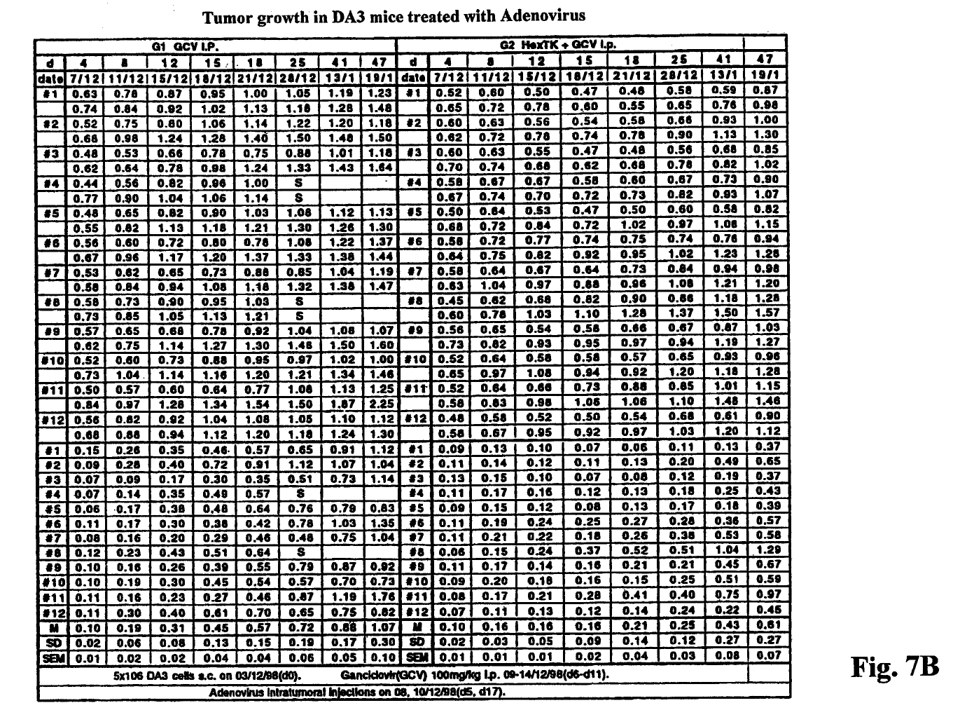

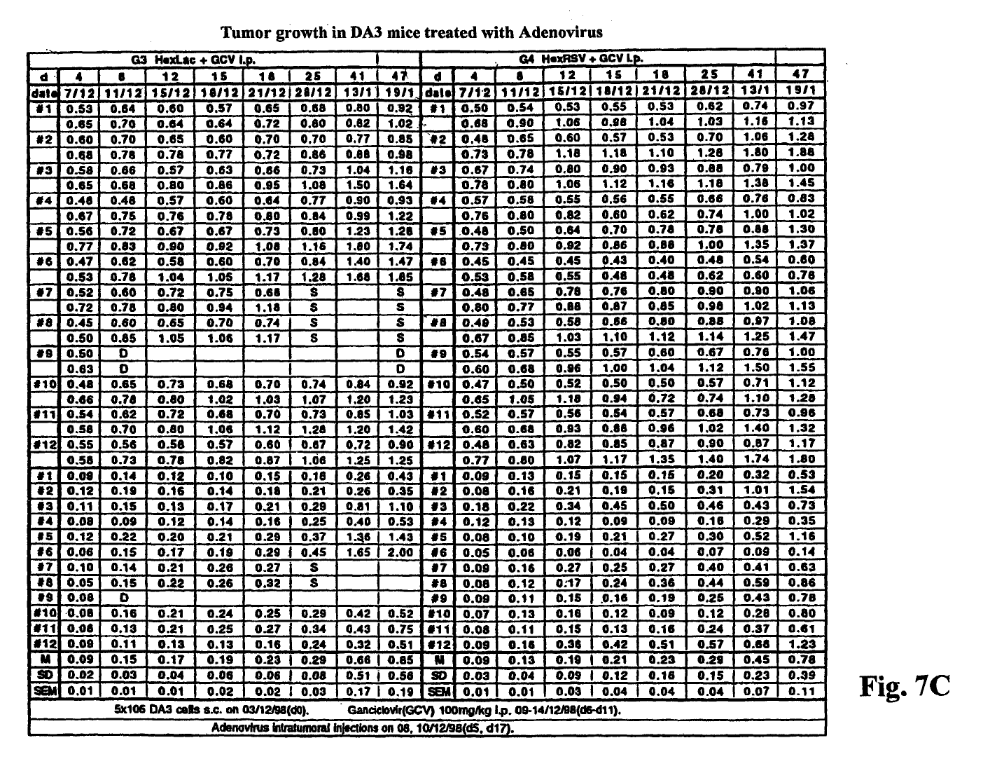

Cell survival in the transfectants exposed to gancyclovir (GCV) at doses of 10 or 25 ug/ml was 50% less than control cells treated with GCV alone or transfected with the plasmid only. FIGS. 7A-7C illustrate results obtained with HexTK, HexLac and RSVTK in recombinant Adenovirus in the treatment of DA-3 mice, together with gancyclovir (GCV) according to an embodiment to the present invention. These results illustrate the utility of the Hex II gene constructs according to the present invention in in vivo cancer therapy.

The regulation of this promoter in human tumor cell lines was studied using glucose, insulin, and glucagon. Lack of metabolic repression was confirmed as described by Mathupala, S. P. et al. ((1995) J. Biol. Chem. 270, 16918-16925). In addition, several samples of human tissues were screened with the HK I, HK II, and HK IV cDNAs to evaluate the level of these enzymes in tissues and asses the safety of using this promoter in gene therapy.

We hypothesize that the Hex II promoter, with or without the metabolic manipulation of the normally express enzyme in muscle using glucagon will provide an important degree of selectivity to the anti-tumor effect. This represents a novel use of a selective promoter, taking advantage of its abnormal regulation in tumor cells.

The present invention will be more readily understood by referring to the following examples which are given to illustrate the invention rather than to limit its scope.

### EXAMPLE I

### In vivo Hex II Directed Gene Transfer and Expression

Histochemical Staining for β-galactosidase Expression

Cells were plated at 2×10⁵ in 6 well plates, one day prior to virus infection. They were infected with viruses at a multiplicity of infection (MOI) of 10. Each condition was done in duplicate. After 48 hrs, they were fixed with 1% glutaraldehyde, washed with 0.02% NP40 in PBS and stained for 16 hrs with 5 mM K₃Fe(CN)₆, 5 mM K₄Fe(CN)₆.3H2O, 0.01% sodium deoxycholate, 2 mM MgCl₂, 1 mM EGTA, 1 mg/ml X-gal in 0.02% NP40 PBS. Staining was scored by visualization under light microscopy.

For in vivo marking studies, two different mouse models were used. For the mouse mammary carcinoma model, DA-3 cells were grown as a subcutaneous tumor in syngeneic mice. For the human lung carcinoma model, NCI-H661 cells were grown as a subcutaneous tumor in nude mice. Cells were trypsinized and washed in ice-cold serum free medium less than 30 minutes before injections. 5×10⁶ cells, resuspended in 0.5 ml of medium were injected subcutaneously in the flank of the animals. Once tumors were palpable, after 3-4 days on average, intratumoral injections of 1×10⁹ plaque forming units (PFUs) of AdHexLacZ or AdΔE1E3 were done with a 25-gauge needle in a total volume of 0.2 ml, in viral storage buffer, at the same injection site. Two mice were injected with each virus, for each model. Animals were sacrificed 48 hrs after viral injections and tumors were excised and snap frozen in liquid nitrogen. Frozen sections of these tumors were mounted on microscope slides and fixers and stained with X-gal as described.

In vivo Studies of HK II-directed Gene Transfer

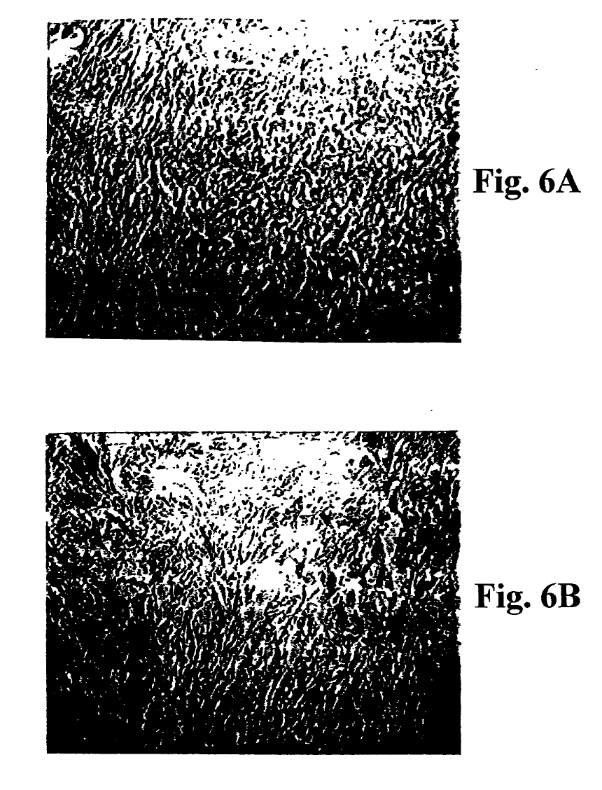

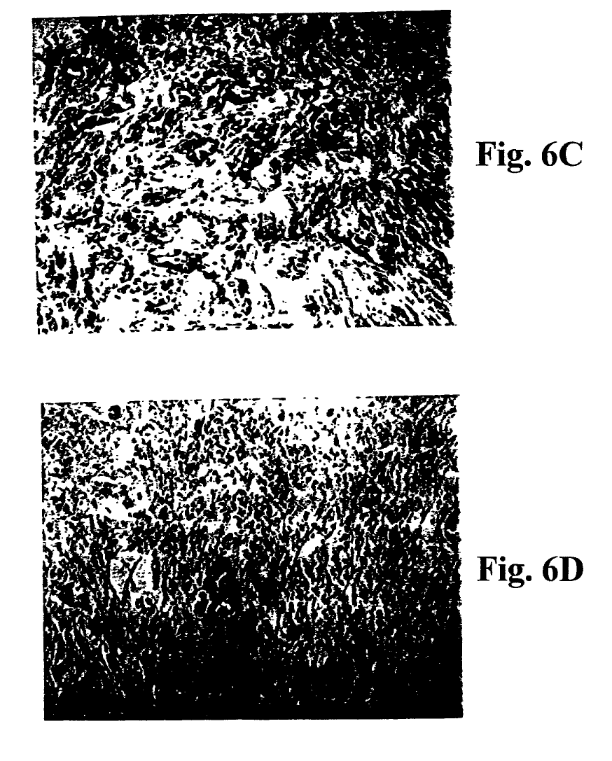

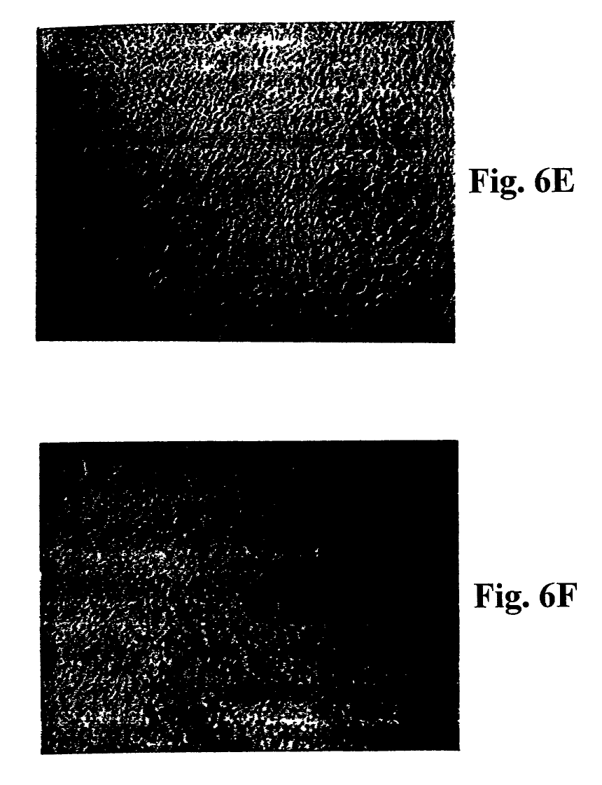

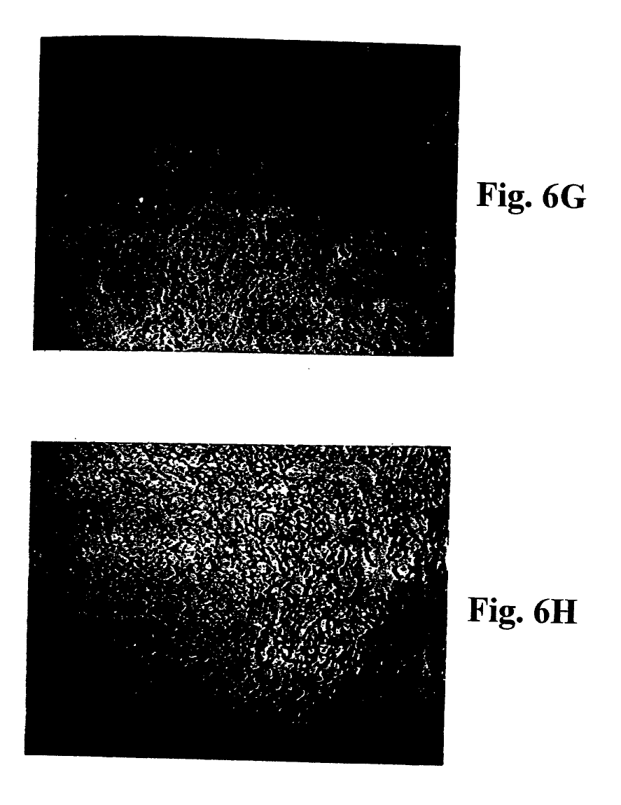

To validate the use of recombinant AdHexLacZ as an efficient marking tool for gene transfer studies in vivo, intratumoral injections were done in the DA-3 and NCI-H661 subcutaneous tumor models. Tumors were injected with AdHexLacZ or AdΔEIE3, which was used as a negative control, and frozen sections of tumor samples were stained with X-gal. In the DA-3 model, AdHexLacZ injected samples showed a patchy X-gal staining (FIG. 6A). In the H661 model (FIG. 6E), the same pattern was observed, and staining represented 10-20% of the tumor sample. Tumors from both models (FIGS. 6A and 6E) injected with the AdΔE1E3 virus did not show any positive staining with X-gal. Hematoxylin and eosin staining of the same samples shows cellular content of the sections (FIGS. 6C, 6D, 6G and 6H). Thus indicating the selective expression of the rat Hex II promoter in the human and mouse tumor cells of these models. Further, as illustrated in FIG. 7A, tumor growth was markedly reduced in DA-3 mice treated with gancyclovir (GCV)and AdHexTK, AdHexLac and AdHexRSVTK, respectively.

### EXAMPLE II

### Use of Hexokinase Type II Promoter in Targeted Gene Therapy for Suicide Destruction of Tumors

The ability to selectively target tumor cells with suicide genes provides an attractive approach for both regional and system cancer therapies. In particular, tumor-selective activation of a suicide gene or the like provides a targeted effect in tumor cells and thereby minimizing the toxicity experienced by normal tissue. The Hex II constructs of the present invention are intended to be used in a vector/delivery system in clinical trials eventually.

Cell Culture

The human breast carcinoma cell lines MCF-7 and ZR-75-1, and NSCLC cell lines NCI-H460 and NCI-H661 (American Type Culture Collection [ATCC], Rockville, Md.), were maintained in RPMI 1640 supplemented with 10% heat-inactivated fetal bovine serum (RPMI 10%). HepG2 (ATCC, Rockville, Md.), a human hepatocellular carcinoma, was propagated in a-MEM 10%. Normal human mammary epithelial cells (NHMECs) and normal human bronchial epithelial cells (NHBECs) are primary cultures grown in MEGM and BEGM, respectively (Clonetics, San Diego, Calif.). Several sets of NHBECs from different donors were tested. MGH-7, a poorly differentiated squamous cell carcinoma cell line established from human primary NSCLC tumors (Liu and Tsao, 1993) was maintained in ACL-4 serum-free medium. H59, a liver metastatic subline of the Lewis lung carcinoma 3LL, is a generous gift from P. Brodt (Department of Surgery, McGill University, Montreal. QC, Canada). It was maintained in RPMI 10%. All media and serum were bought from Mediatech (Herndon, Va.).

Construction of Recombinant Plasmids and Adenovirus

All reporter gene assays were performed using the promoterless chloramphenicol acetyltransferase plasmid vector pCAT basic (Promega, Madison. Wis.). pHexII5150 CAT was constructed by inserting an XbaI cassette derived from the 29-1/Xba(pUC18) plasmid (Mathupala et al., 1995), a generous gift from P. Pedersen (Johns Hopkins University, Baltimore, Md.) in pCAT. The coding region and the first intron present in the XbaI cassette were removed from pHexII5150 CAT with a BamHI digest and religated to generate the pHexII4557 CAT vector (FIG. 1). The XbaI-BamHI cassette of pHexII4557 CAT was inserted in the corresponding sites of pUC18 (Pharmacia Biotech. Baie d'Urfe, QC, Canada) to generate pUC/HexII4557. The BamHI cassette of the β-galactosidase-coding region release from pSNS518 (Genomics One, Montreal, QC, Canada) was inserted into the BamHI site of pUC/HexII4557 to generate pUC/HexII/LacZ. Plasmid DNA was purified with the Maxiprep kit from Qiagen (Santa Clarita. Calif.). pMUC1-1583 CAT contains the mucin-1 promoter and is a gift from J. Taylor-Papadimitriou (Imperial Cancer Research Fund. U.K.). The HindIII-KpnI cassette from pUC/HexII/LacZ was ligated into the corresponding sites of the transfer vector pAdBN (Quantum Biotechnologies. Montreal, Canada), in antisense orientation to the left inverted terminal repeat (ITR). The construct was linearized with AseI and cotransfected with Ad5 ΔE1/E3 linear viral DNA in 293 cells (Quantum Biotechnologies). A recombinant virus was selected after two rounds of plaque purification and purified by cesium chloride gradient. Ad-CMVLacZ is a gift from B. Massie (BRI, Montreal. Canada) and AdΔE1E3 is a gift from F. L. Graham (McMaster, Hamilton, Canada). Viruses were stored at −80° C. in 10% glycerol.

Transfections and Reporter Gene Assays

Transient transfections were performed using Lipofect AMINE (Canadian Life Technologies. Burlington, ON, Canada). Seventy to 80% confluent cells were transfected with 1 μg of the reporter plasmid pCAT, pMUCI-1583 CAT, pHexII4557 CAT, or pRSV CAT (Rous sarcoma virus long terminal repeat promoter) (ATCC). One microgram of pSV₂-Pgalactosidase (Promega) was cotransfected as an internal control for variations in transfection efficiency in serum-free Dulbecco's modified Eagle's medium (DMEM). The medium was replaced with fresh growth medium after 6 hr. In promoter regulation experiments, the cells were transfected in RPMI 1640 glucose-deficient medium. Six hours after transfection, the medium was replaced with RPMI 1640 glucose-deficient medium supplemented with 100 nM bovine insulin. 25 mM glucose, or 10 μM glucagon (Sigma Aldrich Canada. Oakville. ON, Canada) or a combination of insulin and glucose, supplemented with 1 mM sodium pyruvate and 100 μM glucose. Cells were harvested after 24 to 48 hr and lysed in 0.25 M Tris-HCl, pH 7.5. by three successive freeze-thaw cycles. The protein concentration of the extracts was measured using the Bradford protein assay from Bio-Rad (Mississauga, ON. Canada) and equal amounts were assayed in the chloramphenicol acetyltransferase (CAT) and β-galactosidase assays, which were carried out as described by the manufacturer (Promega). Percent acetylation was measured using the Bio-Rad Gelscan Phos-phoimager and the Molecular Analyst software program.

Total RNA Extraction

For regulation studies, cells were grown to subconfluence in 75-cm² flasks, starved in serum-free, glucose-deficient RPMI 1640 medium (Canadian Life Technologies) for 18 hr, and induced in 25 mM glucose, 100 nM insulin, and 10 μM glucagon (Sigma Aldrich Canada) for 12 hr. RNA was extracted from cells using RNAzol (Tel-Test, Friendswood, Tex.) as described by the manufacturer. Contaminating DNA was digested with 5-10 units of DNase I (Pharmacia Biotech) in 10 mM MgCl₂, 1 mM dithiothreitol (DTT), 2010 units of RNA Guard (Pharmacia Biotech), 50-80 μl of total RNA at 1 μg/μl for 30 min at 37° C., followed by precipitation of total RNA.

Ribonuclease Protection Assay

The HK II probe (0.1 fmol) and the human β-actin probe (2 fmol) were simultaneously hybridized to 30 μg of total RNA using the RPA II kit (Ambion, Austin, Tex.) according to the manufacturer recommendations. Probes were prepared by in vitro transcription using the MAXIscript kit (Ambion) and [α-³²P]UTP (6000 Ci/mmol, 40 mCi/ml). HK II (nucleotides 1500 to 1803 of HK II cDNA; GenBank Z-16376) was inserted in the pDP18-T7/T3 vector (Ambion). The template was cut with KpnI at the 5′ end of the insert and transcribed toward that site from the T7 promoter to produce a 430-bp transcript. The HK II-protected fragment is 303 bp. The human β-actin template pTRI-Bactin human was purchased from Ambion and transcribed with the T7 polymerase to produce a 300-bp transcript that protects a 245-bpy fragment. The Ambion Century marker templates were used to synthesize 100- to 500-bp-long fragments. Electrophoresis was performed in 5% acrylamide/8 M urea gels. Quantitation was performed using the NIH Image 1.67b densitometry software.

Histochemical Staining for β-galactosidase Expression

Cells were infected with viruses at a multiplicity of infection (MOI) of 10. After 48 hr, they were fixed with 1% glutaraldehyde, washed with 0.02% Nonidet P-40 (NP-40) in phosphate-buffered saline (PBS), and stained for 16 hr with 5 mM K₃Fe(CN)₆, 5 mM K₄Fe(CN)₆-3H₂O, 0.01% sodium deoxycholate, 2 mM MgCl₂, 1 mM EGTA, 5-bromo-4-chloro-3-indolyl-p-D-galactopyranoside (X-Gal, 1 mg/ml) in 0.02% NP-40-PBS. Staining was scored by visualization under light microscopy.

Killing Cures, Viral Dilutions, and MTT Assays

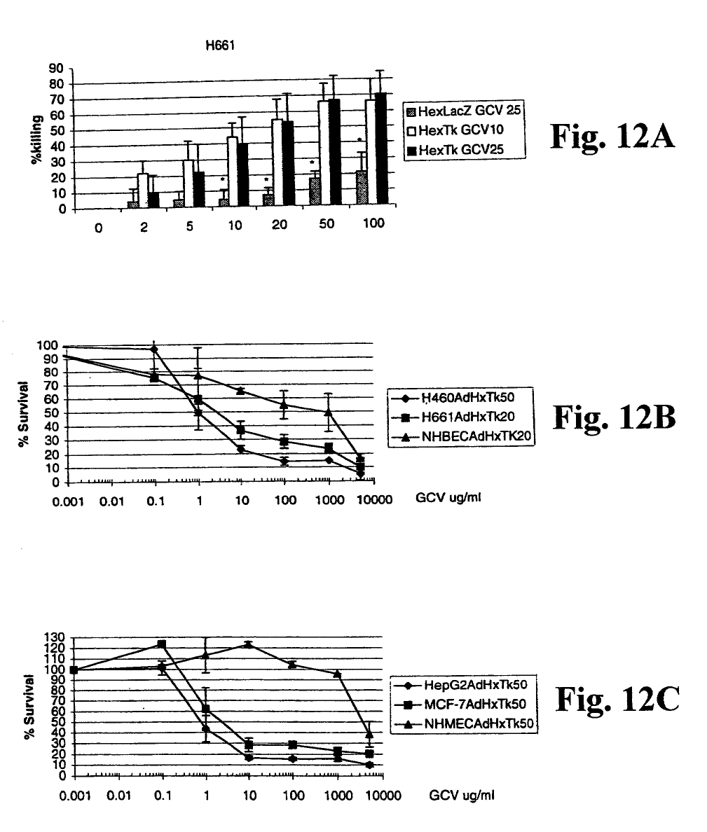

Cell survival was determined using a colorimetric assay that measures the ability of viable cells to reduce a soluble yellow tetrazolium salt (MTT) to an insoluble purple formazan precipitate. The MTT assay was carried out as described (Plumb et al., 1989; Greenbaum et al., 1994). To generate dose-response curves, cells were seeded in 96-well plates at a cell density of 3-5×10³, depending on the cell line, on day 1. On day 2, they were infected with viral dilutions ranging between an MOI of 0 and 100, with either AdHexLacZ or AdHexTk. Ganciclovir (GCV) was added at a concentration of 25 μg/ml for the AdHexLacZ control and at either 10 or 25μ/ml for the AdHexLacZ-infected cells. On day 4, the medium was replaced with fresh medium, along with a new dose of GCV. On day 6, an MTT assay was performed to measure cell viability. Each cell line was tested at least three times. Percent survival is determined by ratios of absorbance values from test conditions over absorbance values from noninfected cells (MOI 0). For killing curves, cells were seeded in 25-cm² flasks on day 1. On day 2, they were infected with the appropriate MOI (see Results, and FIG. 12) with AdHexLacZ or AdHexTk. On day 3, they were trypsinized, counted, and plated at 5×10³ cells/well in 96-well plates. GCV was added at concentrations ranging from 0 to 5000 μg/mI. On day 5, the medium was replaced with fresh medium, along with a fresh dose of GCV. On day 7, the MTT assay was carried out as described above, and cell survival percentages were derived as described above.

Statistical Analysis

One-factor analysis of variance (ANOVA), t tests, with Bonferroni's correction for multiple comparisons and standard deviation were calculated for the appropriate experiments, using Microsoft Excel.

Results

Relative Activity of the HK II Promoter in Normal and Tumor Cells

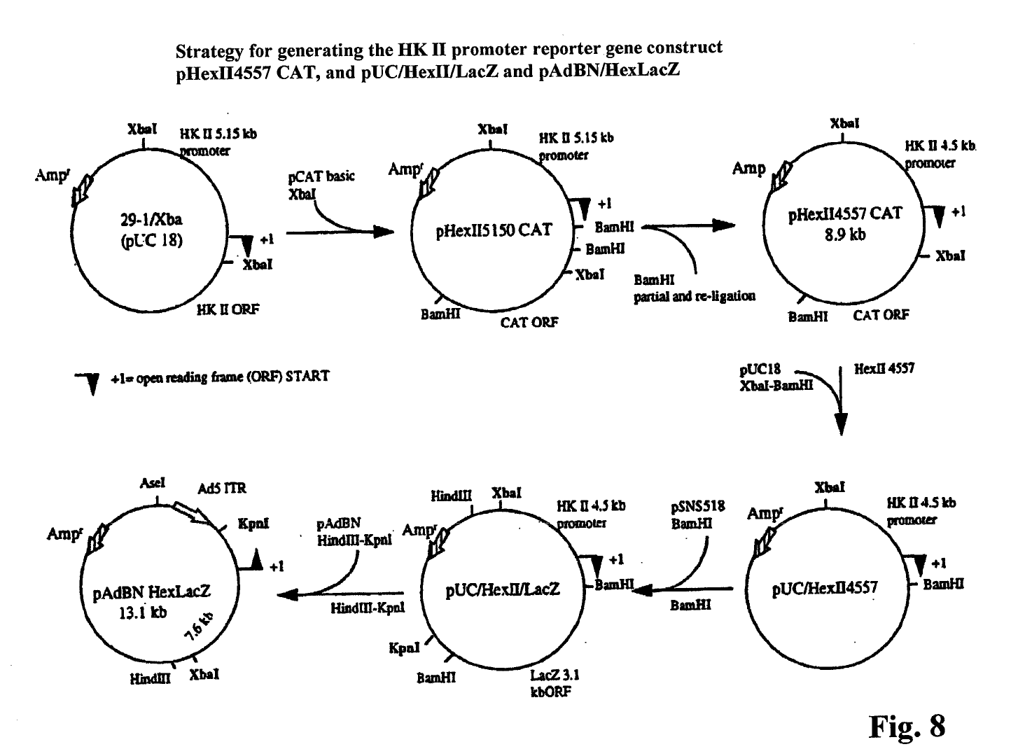

The rat hexokinase type II promoter was subcloned in the pCAT basic vector, in order to study promoter activation using the chloramphenicol acetyltransferase reporter gene (CAT) (FIG. 8). The rat Hex II promoter of the present invention is herein exemplified with a variety of delivery systems for the purpose of illustrating the tumor selective activity thereof. The present invention is not however limited to the delivery systems herein described but may be provided with any suitable delivery system know in the art.

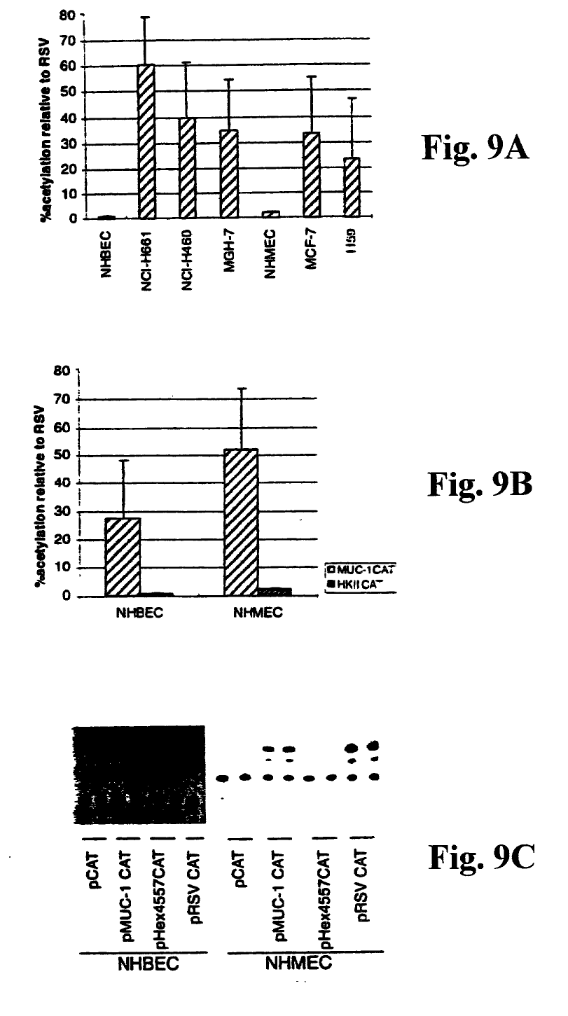

The pHexII4557 CAT reporter gene construct was transiently transfected in a panel of normal and tumor human cells to determine the level of activation of the hexokinase type II promoter. To allow for comparisons among the various cell lines, acetylation in pHexII4557 CAT-transfected cells was corrected for background acetylation observed in cells transfected with pCAT alone. Acetylation values were normalized for variations in transfection efficiency using β-galactosidase activity. The same amount of protein was used for both CAT and β-galactosidase assays, making it unnecessary to express values relative to milligrams of protein. Finally, acetylation in pHexII4557 CAT transfectants is expressed relative to pRSV CAT transfectants (FIG. 9C). FIG. 9C represents the results of a CAT assay in normal cells with the background acetylation from pCAT transfectants subtracted and results were normalized to β-galactosidase activity used as an internal control. HK II promoter activity was higher in all tumor cell lines tested than in primary normal cells of the same origin (FIG. 9A). In lung-derived cells, relative CAT activity in NCI-H661 cells was the highest at 61% of pRSV CAT, followed by NCI-H460 at 40% and MGH-7 at 35%. In contrast, it was low at 0.9% in NHBECs originating from two different donors. A similar pattern was observed in mammary tissue, where CAT activity was at 20% in MCF-7, as opposed to NHMECs, where it was 2.4% of pRSV CAT activity. In H59 cells, derived from a Lewis lure carcinoma, HK II activation was 30% of pRSV CAT activity (ANOVA p value, 0.04). To confirm the tumor versus normal tissue activation of the HK II promoter we compared it with the tissue-specific mucin-1 promoter (MUC-1), which was studied previously in our laboratory. The differences between the activation of these promoters in primary normal human cells of bronchial or mammary origin is striking (FIGS. 9B and 9C). NHBECs transfected with pMUC1 CAT exhibited a relative activity of 28% while the same cells transfected with pHexII4557 CAT had only 0.9% activity. Similarly, NHMECs transfected with pMUC1 CAT exhibited CAT activity of 52% whereas the same cells transfected with pHEXII4557 CAT had only 0.9% activity. Similarly, NHMECs transfected with pMUC1 CAT exhibited CAT activity of 52% whereas the same cells transfected with pHexII4557 CAT had 2.4% activity. These results show the selectivity of HK II activation in tumor against normal cells.

Modulation of HK II Promoter Activity with Regulators of Glucose Metabolism

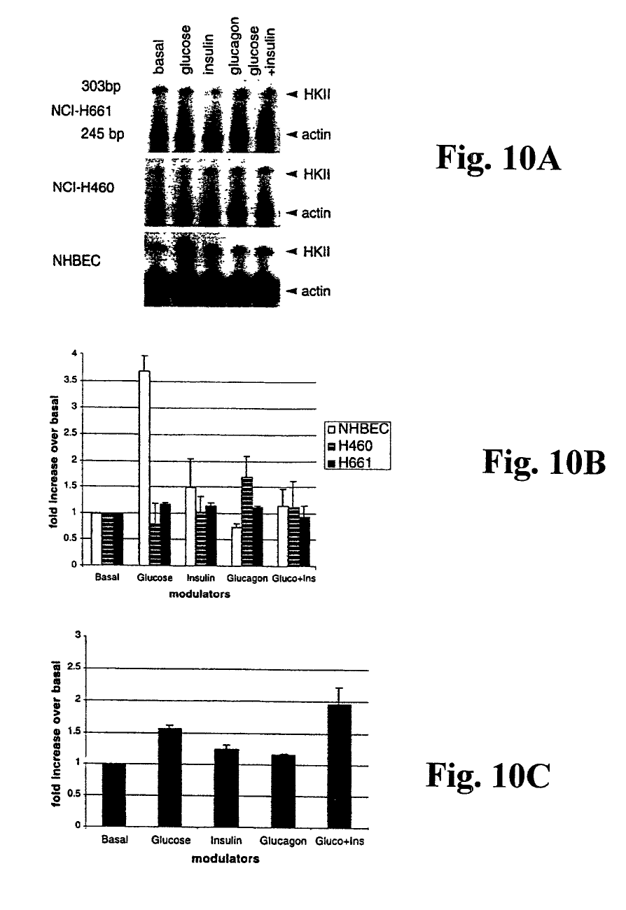

Transcription of the hexokinase type II gene has been studied in rat skeletal muscle tissue and in hepatocytes and was found to be upregulated in response to glucose and insulin and downregulated in response to glucagon (Mathupala et al., 1995). In chemically transformed rat hepatocytes, however, regulation is altered. While insulin and glucose still stimulate HK II activation, the suppression effect of glucagon is lost. Therefore, we were interested to see if this tight regulation was maintained for the rat tumor HK II promoter when transfected in human cells. Regulation of the HK II promoter in normal and transformed cells was investigated. Relative levels of HK II mRNA in NHBECs, NCI-H460, and NCI-H661 were determined (FIG. 10A). Ribonuclease protection assays were performed using simultaneous hybridization with the HK II probe an the human β-actin probe as an internal control. (B) Quantitation of ribonuclease protection assays was also determined as illustrated in FIG. 10B, where values represent HK II mRNA levels normalized to β-actin and expressed as ratios over basal from cells grown under basal conditions (control) or induced with glucose, insulin, glucagon, or a combination of insulin and glucose. Regulation of the HK II promoter was also studied in NCI-H661 cells (FIG. 10C). Cells were transfected with pHexII4557 CAT as described and treated with 25 mM glucose, 100 nM insulin, 10 μM glucagon, or a combination of glucose and insulin. Results were obtained as the average of three independent experiments, with each condition done in duplicate. In human lung carcinoma cell line NCI-H661 (FIG. 10C), promoter activation was greatest with both glucose (25 mMM) and insulin (100 nM), with a two-fold (p=0.03) increase over basal. Induction with glucose, insulin, and glucagon did not lead to significant differences in HK II levels. These results demonstrate the same induction effect of glucose and insulin, but also the same lack of suppressive effect for glucagon observed for the HK II promoter in transformed rat hepatocytes.

Modulation of HK II mRNA Levels in Normal and Tumor Cells

The use of a reporter gene system in transcriptional activation studies to characterize the modulation of HK II in primary NHBECs was not possible since we have shown that NHBECs do not activate HK II to induce CAT activity (FIG. 9B). Instead, endogenous HK II levels in these cells were measured in order to gain insight into the possible mechanism of HK II regulation (FIGS. 10A and 10B). In NHBECs, there was a significant induction over basal levels with glucose (p=0.005), while glucagon repressed HK II expression (P=0.04). This regulation was clearly lost in NCI-H460 and NCI-H661 cells, where levels of HK II mRNA show no significant variation under all conditions. These results demonstrate differences in HK II regulation between human tumor and normal cells, and share features with the previously reported results in rat tissues.

Activation of HK II in Adenoviral Vectors

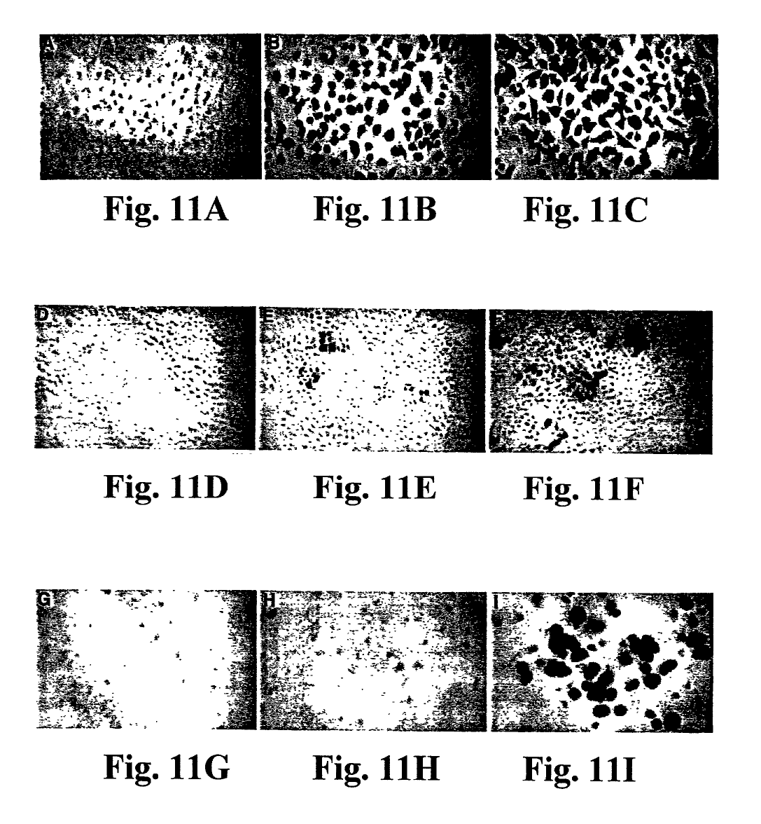

To test the feasibility of using the HK II promoter in adenoviral vectors for targeted gene therapy, AdHexLacZ was used to infect NHBECs, NCI-H460, and NCI-H661. Levels of XGal staining were compared for each cell line with those of Ad-CMVLacZ-infected cells used as a positive control and AdΔEIE3-infected cells as a negative control (FIG. 11). NCI-H661 cells transduced with AdHexLacZ (FIG. 11B) showed the highest level of staining at 95% of a AdCMVLacZ-transduced cells (FIG. 11C), while the level of staining in AdHexLacZ-infected NCI-H460 cells (FIG. 11E) was 10% of AdCMVLacZ-infected cells (FIG. 11F). In NHBECs, staining in AdHexLacZ (FIG. 11H)-infected cells was 1% of AdCMVLacZ-infected cells (FIG. 11I). Staining in NCI-H661, NCI-H460, and NHBECs (FIGS. 11A, 11D and 11G, respectively) infected with AdΔEIE3 was negative.

HK II-directed TK/GCV Killing using Adenoviral Vectors

To control for possible differences between cell lines with respect to adenoviral infectivity, we created dose-response curves. We varied the MOIs of AdHexTk and looked at cell killing at two doses of GCV, 10 and 25 μg/ml, for each of the six cell lines studied. From these experiments, we defined an optimal MOI for each cell line in the linear range of the curve to prevent saturation of infection. The results are summarized in Table 1. The choice was also based on maximal cell killing in AdHexTk-transduced cells, along with minimal toxicity in AdHexLacZ-transduced cells, on GCV treatment. In NCI-H661 cells, shown as an example in FIG. 12A, an MOI of 20 resulted in 7% killing with the AdHexLacZ control while it resulted in 54% killing with AdHexTk (p=0.0025 average of four experiments). Moreover, cell killing continued to increase at an MOI of 50, showing there was no saturation at MOI 20. For NHMECs and NHBECs, there was minimal killing across a 10-fold increase in MOI. Therefore, we selected the MOI value that also resulted in the least toxicity in AdHexLacZ-transduced cells. We have shown that these two cell lines are transducible by adenoviruses by X-Gal staining after AdCMVLacZ infection (data not shown and FIG. 11, respectively). Using the optimal MOI, we performed cell-killing assays over a range of GCV concentrations in AdHexTk-transduced cells. The curves are shown in FIG. 12B and the IC₅₀ values are summarized in Table 1. The results demonstrate selectivity in toxicity, with a 10- to 100-fold increase in IC₅₀ between lung cancer cell lines H661 and H460, respectively, and NHBECs. There was also a 100-fold increase in IC₅₀ in NHMECs relative to breast carcinoma cell line MCF-7. In HepG2 cells, an IC₅₀ of 1 μg/ml was observed, comparable to other tumor cell lines.

TABLE 1
Optimal MOI Values for Cell Lines Studied

| Cell Line | Optimal MOI | IC₅₀ (μg/ml) |
|---|---|---|
| NHBEC | 20 | 100 |
| NCI-H460 | 50 | 1 |
| HCI-H661 | 20 | 1 |
| NHMEC | 50 | >1000 |
| MCF-7 | 50 | 10 |
| HepG2 | 50 | 1 |

### EXAMPLE III

### In vivo Hex II Directed Gene Transfer and Expression

The pΔE1sp1BHex-LacZ and other Hex II gene constructs of the present invention may be used in tumor bearing rats for the in vivo localization of the suicide gene in pre-clinical testing of this novel targeting strategy. The gene construct may be administered in adenovirus type 5 recombinant vector or in a lipid-based delivery system.

Materials and methods

Construction of Recombinant Viruses

Recombinant, replication deficient adenoviral vectors derived from type 5 adenovirus are constructed by the homologous recombination method in the human embryonic kidney cell line 293. The recombinant shuttle plasmids and pBHG11, containing the adenoviral genome, are co-transfected by calcium phosphate precipitation in 293 cells. The viral DNA is isolated from a single plaque and analyzed by restriction enzyme digestion. Recombinant adenovirus is expanded from a single plaque in 293 cells. Large scale production of the recombinant adenovirus is accomplished by growth in 293 spinner cells and purification by double cesium chloride gradient.

Results

According to these experiments, the best method of administration of the gene construct may be determined. It can either be done regionally to target specific organs such as the liver through portal vein injection or it can be administered intravenously. This method of looking at the distribution of the gene will allow us to determine the efficacy of uptake in the various organs and therefore establish a standard for use in humans.

### EXAMPLE IV

### Use of Hexokinase Type II Promoter in Targeted Gene Therapy for Suicide Destruction of Tumors

It is fully contemplated that the above-described HexII/VTK construct will be used in a vector/delivery system in clinical trials eventually. Further, the Hex II promoter of the present invention may be provided in a suitable construct to drive any one of a full range of possible cancer therapeutic genes, including suicide genes such as TK, antisense oligonucleotides, and pro-apoptotic genes in a tumor selective fashion for the purpose of treating cancer.

Discussion

Strong Hex II promoter activation in lung and mammary tumors was achieved by the constructs of the present invention. Further, this Hex II promoter activation was shown to be tumor-specific, by repeating the same experiments in normal primary cells of the same origins. Differential regulation of the Hex II promoter was also demonstrated in human tumor cells with the loss of repression by glucagon.

The results obtained with the Hex II promoter constructs of the present invention in an adenoviral expression system with a β-galactosidase marker gene, confirm the tumor-specificity of the Hex II promoter. In particular, lung tumor cell lines H661 and H460 activated the Hex II promoter, while normal human bronchial epithelial cells (NHBECs) did not. The present invention therefore provides a tumor selective rat Hex II promoter. Although the selectivity of the rat Hex II promoter is herein illustrated with a variety of plasmids and recombinant adenoviruses, the present invention is not limited to a particular delivery system. In fact, the tumor selective promoter of the invention may be delivered to a cell using any suitable gene delivery tool known in the art.

In addition, the utility of the rat Hex II promoter construct(s) of the present invention in gene therapy was confirmed by the tumor-selective cell death achieved when the present invention was employed in a gene-directed enzyme prodrug therapy protocol in combination with a thymidine kinase gene and gancyclovir. This tumor-selective cell death indicates that that the rat Hex II promoter is an ideal candidate as a targeting tool for gene therapy, and in particular, a tool for tumor-specific in vivo cancer therapy.

It should be understood that the rat Hex II promoter construct(s) of the present invention may adapted for use in a suitable delivery system together with a gene for providing tumor selective expression of the gene. For example, for clinical use of the Hex II promoter, it may be preferred to pre-screen tumor samples in order to determine the degree of activation of a Hex II-driven gene. Therefore, according to an embodiment of the present invention there is provided a kit for screening Hex II-driven gene expression in vitro. A kit according to the present invention may include means for culturing a biopsied tumor in vitro and a Hex II promoter reporter gene system for determining Hex II-driven gene expression in tumor cell of the biopsied tumor. In addition, the rat Hex II promoter construct(s) in accordance with the present invention may be provided in the form of a laboratory reagent kit for use in determining selective expression of genetic material in tumor cells as compared to normal cells.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

We claim:

1. A tumor-specific gene construct, which comprises a rat Hex II promoter in a suitable vector, wherein said promoter is selectively activated in tumor cells as compared with normal cells.

2. The gene construct of claim 1, which further comprises LacZ or HSV Tk.

3. The gene construct of claim 1, wherein said vector is selected from one of a basic expression vector, a shuttle plasmid, an adenovirus type 5 recombinant vector or a lipid-based delivery system.

4. A vector for use in selective gene expression in a tumor cell, said vector comprising a rat Hex II promoter that is selectively activated in tumor cells as compared with normal cells.

5. A method for a tumor-selective expression of a gene in a cell comprising inserting in said cell a gene construct comprising said gene operably linked to a tumor-specific rat Hex II promoter, whereby said rat Hex II promoter is selectively activated in tumor cells as compared with normal cells.

6. The method of claim 5, wherein said gene construct is inserted in said cell in vitro.

7. The method of claim 5, wherein said gene construct is inserted in said cell in vivo.

8. The method of claim 5, wherein said gene construct is inserted in an adenovirus type 5 recombinant vector or in a lipid-based delivery system.

9. The method of claim 5, wherein said gene encodes an enzyme that converts an otherwise non-toxic prodrug into its active form, wherein said method further comprises exposing said tumor.

10. The method of claim 9, wherein said gene is HSV Tk or Cytochrome P-450™ 2B1.

11. The method of claim 10, wherein said gene is HSV Tk and wherein said prodrug is ganciclovir.

12. The method of claim 10, wherein said gene is Cytochrome P-450™ 2B1 and wherein said prodrug is cyclophosphamide, penicillin, amidase or β-lactamase.

13. The gene construct of claim 1, wherein said construct is pHexII4557-CAT as set forth in FIG. 1.

14. The tumor-specific Hex II gene construct of claim 2, wherein said tumor cells are human tumor cells.

15. A tumor-specific Hex II gene construct comprising a rat Hex II promoter operatively linked to a gene and a vector selected from one of a basic expression vector, a shuttle plasmid, an adenovirus type 5 recombinant vector or a lipid-based delivery system.

16. The tumor-specific Hex II gene construct of claim 15, wherein said rat Hex II promoter is provided in a negative orientation relative to a polycloning site of said vector.

17. The tumor-specific Hex II gene construct of claim 3 for use in selective expression of a gene in human tumor cells.

18. The tumor-specific Hex II gene construct of claim 15 for use in selective expression of a gene in non-human tumor cells.

19. The gene construct of claim 15, wherein said vector is p E1sp1B and said construct is p E1sp1BHex-LacZ as set forth in FIG. 2.

20. The gene construct of claim 15, wherein said vector is p E1sp1B and said construct is p E1sp1BHex-TK as set forth in FIG. 3.

21. A kit adapted to provide the tumor-specific gene construct of claim 1.

22. The kit of claim 21 for use in screening tumor specific gene expression in vitro.

23. The gene construct of claim 3, wherein said vector is an AdHexTK.

24. The gene construct of claim 3, wherein said vector is AdHexLacZ.

25. The gene construct of claim 3, wherein said vector is pAdBN/HexLacZ.
